(19) United States
(12) Patent Application Publication
(10) Pub. No.: US 2004/0193020 A1
(43) Pub. Date: Sep. 30, 2004

(54) IN-VIVO INFORMATION EXTRACTING SYSTEM, TAG DEVICE USED FOR THE SAME, AND RELAY DEVICE

(75) Inventors: Toshio Chiba, Tokyo (JP); Susumu Kusakabe, Kanagawa (JP)

Correspondence Address: CONNOLLY BOVE LODGE & HUTZ LLP, SUITE 800, 1990 M STREET NW, WASHINGTON, DC, 20036-3425, US

(73) Assignee: REAMETARU CO., LTD., Tokyo (JP)

(21) Appl. No.: 10/707,960

(22) Filed: Jan. 28, 2004

Related U.S. Application Data
(63) Continuation of application No. PCT/JP02/08569, filed on Aug. 26, 2002.

(30) Foreign Application Priority Data
Aug. 29, 2001 (JP) JP2001-259516
Dec. 6, 2001 (JP) JP2001-372861

(51) Int. Cl. A61B 5/00

(52) U.S. Cl. 600/300; 128/903

(57) ABSTRACT

An in-vivo information extracting system comprises a tag device (1) embedded in a living body, a relay device (2) disposed outside the living body and near the tag device (1), and main transceiver (3) for collecting the in-vivo information extracted by the tag device (1) through the relay device (2). The tag device (1) has a rectifier circuit for rectifying an electromagnetic wave received from the relay device (2) and generating an operating power. By thus generating, inside the tag device, the operating power needed to drive the tag device (1) from the electromagnetic wave fed from outside by means of an RFID, any cell or battery in the tag device (1) is not needed, and the size of the tag device (1) is accordingly reduced. Therefore, the tag device can be used in a living body almost permanently.

### CROSS REFERENCE TO RELATED APPLICATIONS

This Application is a Continuation of application PCT/JP02/08569 filed on Aug. 26, 2002, the entire contents of which are incorporated herein by reference. PCT/JP02/08569 claims priority to Japanese application 2001-372861 filed on Dec. 6, 2001, and claims priority to Japanese application 2001-259516 filed on Aug. 29, 2001.

### BACKGROUND

The present invention relates to an in-vivo information extracting system as well as to a tag device and relay device used therefor. In particular, it is suitable for a system which extracts various in-vivo information using a tag device embedded in the body of a person or animal and takes the information wirelessly out of the body.

Recently, tag devices which can be embedded or left in the bodies of humans or animals have been used for medical services to detect various in-vivo information. For example, tag devices are equipped with a temperature sensor and pressure sensor, so that they can detect body temperature, blood pressure, etc. within the living body and send the acquired information wirelessly out of the body.

FIGS. 1A and 1B are diagrams showing a prior art example of a medical tag device. Of these, FIG. 1A shows an external configuration of the medical tag device. As shown in the figure, the medical tag device comprises a battery 52 and circuit board 53 both enclosed in a capsule 51 made of plastics or the like. The battery 52 and circuit board 53 are connected electrically with each other so that the circuit board 53 draws its operating power from the battery 52.

FIG. 1B shows major circuits on the circuit board 53. In the figure, a control circuit 61 performs overall control of the medical tag device as well as data processing. A sensor 62 may be a temperature sensor or pressure sensor, for example, and detects body temperature or blood pressure within the living body embedded with the tag device. Output signals from the sensor 62 are input in the control circuit 61, where they undergo predetermined data processing such as binarization.

A memory 63 prestores data needed for operation of the medical tag device and consists, for example, of an EEP-ROM. A modulator 64 modulates a signal to be transmitted into a format suitable for transmission by way of ASK (Amplitude Shift Keying) or FSK (Frequency Shift Keying) and supplies the resulting signal to a transmission antenna 65. The modulated in-vivo information is sent from the transmission antenna 65 to an external information processing unit.

The conventional medical tag device described above must contain the battery 52 to provide operating power to the circuit board 53. This increases the size of the medical tag device. In particular, it requires a large amount of transmission power to transmit in-vivo information out of the body, increasing the size of the battery 52 naturally. Medical tag devices currently in use are as large as approximately 10 mm in capsule 51 diameter and approximately 40 mm in capsule length. This has the problem of causing inconvenience, discomfort, or pain to the people or animals embedded with the medical tag devices.

Also, there is the problem that the medical tag devices cannot be left in the living body for long-term use because the battery 52 has a service life. To collect in-vivo information on a continuous basis for use in treatment, health care, etc. it is desirable to leave and use medical tag devices in the living body for extended periods of time. However, conventional medical tag device, which becomes unserviceable when the battery 52 is low, need to be removed from the body and embedded anew.

Some medical tag devices supply operating power to the battery 52 inside the body from an external power source outside the body by using a rechargeable secondary cell as the battery 52 and passing a detachable lead wire through capsule 51. However, this prior art example also has the problem of causing inconvenience or great pain because it inserts the lead wire into the living body to recharge the battery 52 contained in the capsule 51.

The present invention has been made to solve the above problem and has an object to make it possible to downsize tag devices and reduce inconvenience, discomfort, and pain caused to the living bodies embedded with them.

Another object of the present invention is to make it possible to leave and use tag devices in living bodies for an extended period of time without concern for battery life.

### BRIEF SUMMARY

An in-vivo information extracting system according to the present invention comprises: a tag device which extracts in-vivo information in a living body; and a relay device which is installed outside the living body and near the tag device embedded in the living body, characterized in that the tag device comprises power generating means for generating internal operating power from an electromagnetic wave fed from outside the tag device, and the relay device comprises transceiver means for receiving, from the tag device, the in-vivo information extracted by the tag device and transmitting the received in-vivo information to outside the relay device.

According to another aspect of the present invention, the relay device comprises a power supply as a source of operating power for the transceiver means.

### DESCRIPTION OF THE DRAWINGS

FIGS. 1A and 1B are diagrams showing a prior art example of a medical tag device;

FIG. 2 is a diagram showing an overall configuration example of an in-vivo information extracting system according to this embodiment;

FIG. 3 is a diagram showing a configuration example of a tag device according to this embodiment;

FIGS. 4A, 4B, 4C, and 4D are diagrams showing formation examples of a planar loop antenna used in this embodiment; and

FIG. 5 is a diagram showing a configuration example of a relay device according to this embodiment.

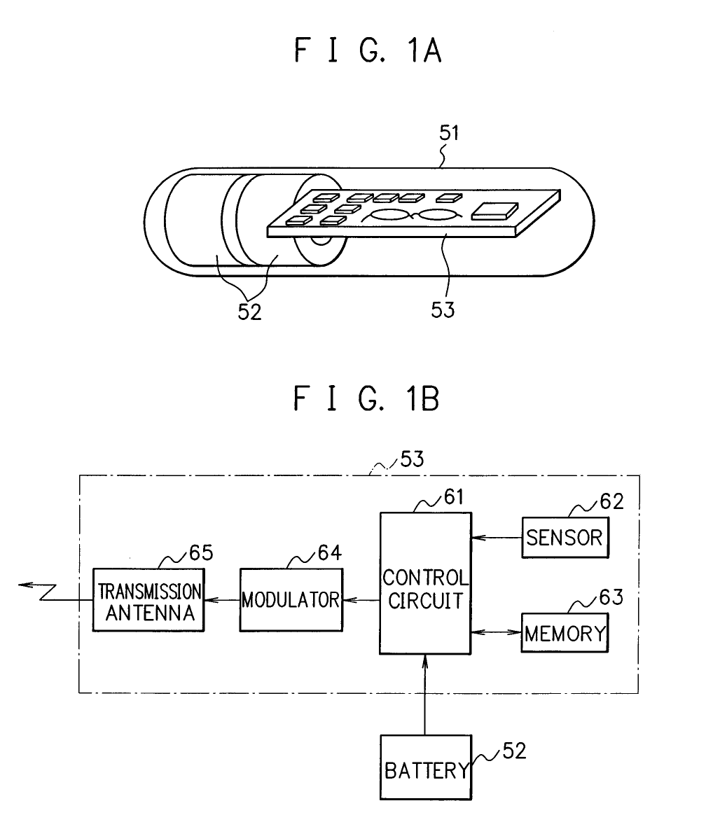

### DETAILED DESCRIPTION

An embodiment of the present invention will be described below with reference to the drawings.

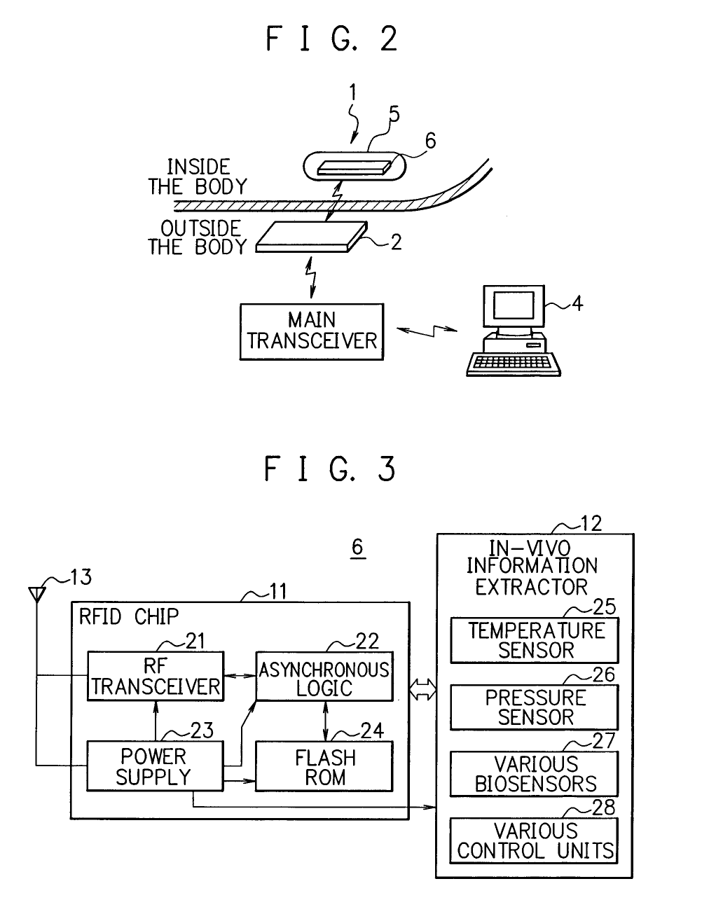

FIG. 2 is a diagram showing an overall configuration example of an in-vivo information extracting system according to this embodiment. As shown in the figure, the in-vivo information extracting system according to this embodiment comprises a tag device 1 used by being embedded in, or swallowed into, a human or animal body, a relay device 2 installed outside the body and near the tag device 1 placed in the body, a main transceiver 3 which transmits and receives signals to/from the relay device 2, and an information processing unit 4 which collects and processes in-vivo information extracted by the tag device 1.

The tag device 1 comprises a transmit/receive antenna and very small module board 6 both enclosed in a capsule 5 made of plastics or the like. The module board 6 is equipped with an RFID (Radio Frequency Identification) chip, various application-specific sensors, etc. as described later. The RFID chip is equipped with a RF transceiver to transmit and receive radio-frequency signals (RF signals) to/from the relay device 2. The tag device 1 is used, being left at a desired site within the living body.

The relay device 2 comprises a transmit/receive antenna and module board. The module board is equipped with a RF transceiver for sending and receiving (relaying) RF signals to/from the tag device 1 and main transceiver 3, IC chip for performing predetermined data processing, etc. as described later. The RF transceiver also serves to send an electromagnetic wave (radio wave) for providing an electromotive force to the tag device 1.

The relay device 2 is used, being installed outside the body and near the tag device 1 left in the body. For example, it is installed on the patient's bed or on an MRI (Magnetic Resonance Imaging), CT (Computerized Topography) scanning, NMR (Nuclear Magnetic Resonance), or other apparatus for clinical examination. It may also be fastened to the surface of the body with adhesive tape, a bandage, or a belt or may be attached to the patient's clothes. This allows the patient to move freely because there is no need for the patient to stay near the place where the relay device 2 is fastened. In short, the relay device 2 may be installed in any manner as long as it is close enough to the tag device 1 to be able to communicate with the latter.

The main transceiver 3 exchanges necessary data with the relay device 2. According to this embodiment, in particular, the main transceiver 3 receives commands from the information processing unit 4 and transmits them to the tag device 1 in the body via the relay device 2 installed in the vicinity as well as receives the in-vivo information extracted by the tag device 1 via the relay device 2 and sends it out to the information processing unit 4.

The information processing unit 4 consists, for example, of a personal computer (PC). It generates various commands for controlling the tag device 1 in the body and outputs them to the main transceiver 3 as well as utilizes the in-vivo information collected by the tag device 1 for treatment of living bodies, diagnosis, disease control, health care, medical researches, ecological surveys, etc. by displaying it, analyzing it, etc. Incidentally, although the main transceiver 3 and information processing unit 4 are configured separately, the information processing unit 4 may incorporate a function of radio communication of the main transceiver 3.

FIG. 3 is a diagram showing a configuration example of the tag device 1. As shown in the figure, the module board 6 of the tag device 1 is equipped with an RFID chip 11 and in-vivo information extractor 12. Also, the RFID chip 11 on the module board 6 is electrically connected with a transmit/receive antenna 13.

The transmit/receive antenna 13 transmits and receives RF signals, for example, in the range of a few MHz to 2.45 GHz or 5.75 GHz. It consists, for example, of a radio-frequency planar loop antenna to reduce the size of the tag device 1. Incidentally, although the transmit/receive antenna 13 in FIG. 3 is configured as a single antenna, a transmit antenna and a receive antenna may be provided separately.

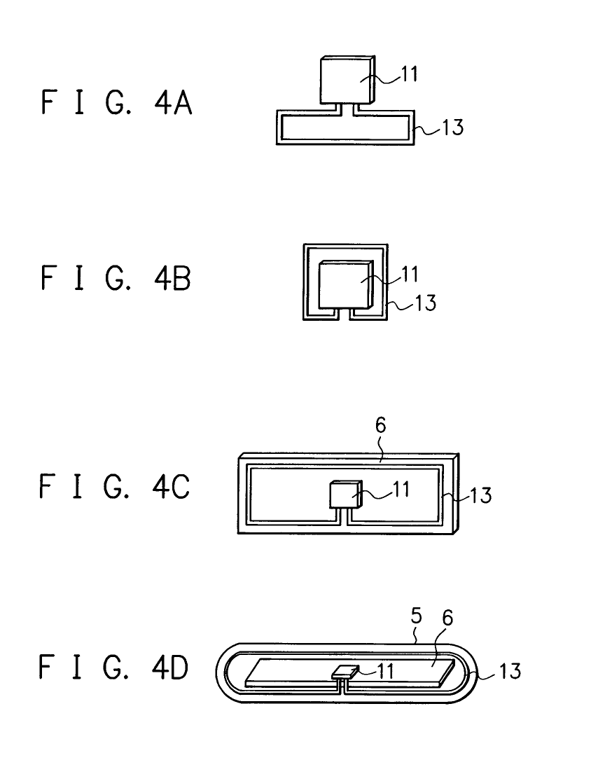

FIGS. 4A, 4B, 4C, and 4D show formation examples of a planar loop antenna 13. FIG. 4A shows an example in which the planar loop antenna 13 is formed in a different area of the module board 6 from the RFID chip 11. FIG. 4B shows an example in which the planar loop antenna 13 is formed around the RFID chip 11. FIG. 4C shows an example in which the planar loop antenna 13 is formed around the RFID chip 11 as is the case with FIG. 4B, but the planar loop antenna 13 is mounted on the module board 6 as a printed pattern.

FIG. 4D shows an example in which the planar loop antenna 13 is formed around the module board 6 on which the RFID chip 11 is mounted. For example, the planar loop antenna 13 can be formed on the surface of the capsule 5 by metal printing. Also, it is possible to use the capsule 5 itself, i.e., the container of the tag device 1, as a radio-frequency antenna instead of installing a particular planar loop antenna 13. In radio-frequency areas, parasitic elements or floating elements will be produced and especially in the case of a small device, the container itself will act as a path for current. Thus, the capsule 5 itself can be used as a radio-frequency antenna when conditions are right, such as when the capsule 5 is made of a high di-electric material.

If the planar loop antenna 13 is formed as shown in FIG. 4A, the size of the tag device 1 is increased by the area occupied by the planar loop antenna 13. In contrast, if the planar loop antenna 13 is formed as shown in FIGS. 4B to 4D, it is possible to avoid a situation in which the size of the tag device 1 is increased by the area occupied by the planar loop antenna 13. Especially, the use of the capsule 5 as a radio-frequency antenna will eliminate the need to install the planar loop antenna 13, making it possible to further reduce the size of the tag device 1.

Incidentally, although the planar loop antenna 13 is used here, it goes without saying that another type of radio-frequency antenna may be used. Also, although the planar loop antenna is used here as the transmit/receive antenna 13 because radio-frequency signals are used for communications between the tag device 1 and relay device 2, a coil antenna made of a coiled conductor may be used if low-frequency signals below 1 MHz (e.g., 140 KHz) are used for the communications.

Returning to FIG. 3, description will be given of configuration of the RFID chip 11 and in-vivo information extractor 12. The RFID chip 11 is equipped with an RF transceiver 21, asynchronous logic 22, power supply 23, and flash ROM 24. The RF transceiver 21 transmits and receives RF signals to/from the relay device 2 via the transmit/receive antenna 13 on a non-contact basis. The RF transceiver 21 has capabilities, including a modulation capability to modulate signals to be transmitted into a format suitable for transmission by ASK, FSK, or the like and a demodulation capability to demodulate received signals into a format suitable for internal processing by PSK (Phase Shift Keying) or the like.

The asynchronous logic 22 is a signal processor which performs data processing as well as overall control of the RFID chip 11 and in-vivo information extractor 12. For example, it controls the in-vivo information extractor 12 according to commands sent from the information processing unit 4 via the main transceiver 3 and relay device 2. Also, it binarizes measured data of the body's internal environment outputted from the in-vivo information extractor 12 and encrypts data using an encrypted ID stored in the flash ROM 24. The encrypted in-vivo information is modulated by the RF transceiver 21 and then transmitted to the relay device 2 outside the body.

The power supply 23 generates an alternating voltage by electromagnetic induction from an RF signal (electromagnetic wave) sent from the relay device 2 via the transmit/receive antenna 13, rectifies it into a dc voltage, and thereby internally generates operating power needed to drive the RFID chip 11 and in-vivo information extractor 12. The RF transceiver 21 and asynchronous logic 22 described above as well as the flash ROM 24 and the like describe later are driven by the operating power generated by the power supply 23.

The flash ROM 24 is used to store the above-described encrypted ID, attribute information about a living body (personal information about a person), etc. in advance. The information stored in the flash ROM 24 is read by the asynchronous logic 22 and used for processing in the RFID chip 11. Incidentally, although the flash ROM is used here, this is only exemplary and another type of memory such as EEPROM or RAM may be used alternatively.

Also, the in-vivo information extractor 12 comprises a temperature sensor 25, a pressure sensor 26, various biosensors 27, various control units 28, etc. and uses them to measure the environment within the living body. For example, it measures the body temperature, blood pressure, blood sugar level, composition of blood and other body fluids, pH level, pulse rate, heart beats, hardness and viscosity of the inner body wall, light reflex characteristics, etc. in the living body.

It is also possible to pick up images in the body with a small camera which employs a CCD (Charge Coupled Device), CMOS (Complimentary Metal Oxide Semiconductor), or other element or to pick up sounds in the body with a small microphone. When picking up images in the body with a small camera, it is preferable to provide a small illuminating device which illuminates the interior of the body using the electromotive force from the power supply 23. Incidentally, what has been described here is only exemplary, and is not meant to be restrictive.

Such in-vivo information may be gathered either by controlling the in-vivo information extractor 12 according to commands from the information processing unit 4 or by the in-vivo information extractor 12 on its own irrespective of commands from the information processing unit 4. When the commands are used, it is possible, for example, to control the extraction timing or extraction time of in-vivo information, specify the data to be gathered, control the ON/OFF operation of the illumination, control the pan and tilt of the small camera, and so on.

In the configuration of the tag device 1 described above, the RF transceiver 21 and transmit/receive antenna 13 constitute the tag reception means and tag transmission means of the present invention. The power supply 23 constitutes the power generating means of the present invention and the in-vivo information extractor 12 constitutes the in-vivo information extracting means of the present invention. The asynchronous logic 22 constitutes the control means of the present invention.

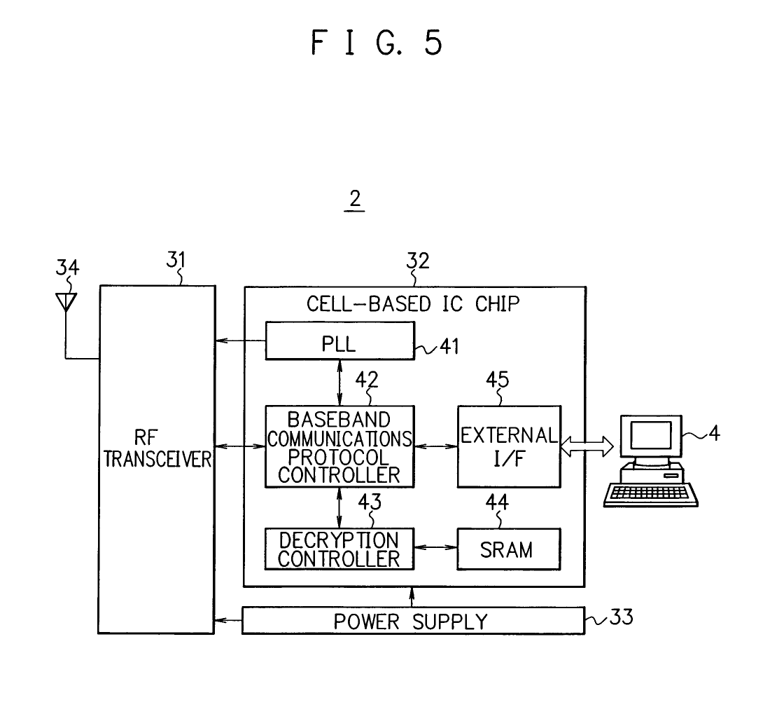

FIG. 5 is a diagram showing a configuration example of the relay device 2. As shown in the figure, the module board of the relay device 2 comprises an RF transceiver 31, cell-based IC chip 32, and power supply 33. The RF transceiver 31 is electrically connected with a transmit/receive antenna 34.

The transmit/receive antenna 34 transmits and receives RF signals, for example, in the range of a few MHz to 2.45 GHz or 5.75 GHz. It consists, for example, of a radio-frequency planar loop antenna. Incidentally, although the transmit/receive antenna 34 in FIG. 5 is configured as a single antenna, a transmit antenna and a receive antenna may be provided separately.

Incidentally, the relay device 2, which is used outside the body unlike the tag device 1 embedded in the living body, needs not be so small as the tag device 1. Therefore, a radio-frequency antenna with a high transmit/receive efficiency than a planar loop antenna may be used. Also, if low-frequency signals are used for communications with the tag device 1, a coil antenna may be used.

The RF transceiver 31 transmits and receives RF signals to/from the tag device 1 and main transceiver 3 via the transmit/receive antenna 34 on a non-contact basis. For example, it transfers commands and other RF signals received from the main transceiver 3 to the tag device 1 in the body as well as transfers in-vivo information and other RF signals received from the tag device 1 in the body to the main transceiver 3. The RF transceiver 31 has capabilities, including a modulation capability to modulate signals to be transmitted into a format suitable for transmission by ASK, FSK, or the like and a demodulation capability to demodulate received signals into a format suitable for internal processing by PSK or the like.

The cell-based IC chip 32 is equipped with a PLL (Phase Locked Loop) circuit 41, baseband communications protocol controller 42, decryption controller 43, SRAM (Static RAM) 44, and external interface 45. The PLL circuit 41 generates and outputs signals with a local oscillator frequency for use in the RF transceiver 31.

The baseband communications protocol controller 42 controls communications between the relay device 2 and tag device 1 as well as between the relay device 2 and main transceiver 3 according to a predetermined communication protocol. Broadly speaking, it controls transmission of an in-vivo information collection request signal and various other commands from the main transceiver 3 to the tag device 1 as well as transmission of in-vivo information sent from the tag device 1 to the main transceiver 3 in response.

The decryption controller 43 decrypts the data encrypted by the tag device 1. During the decryption, it uses the SRAM 44 as working memory. The external interface 45 exchanges various data with the information processing unit 4. Normally, the data exchange between the relay device 2 and information processing unit 4 is performed via the main transceiver 3. In doing that, the communications between the relay device 2 and main transceiver 3 are conducted by the RF transceiver 31 of the relay device 2 on a non-contact basis. In addition, data can be exchanged directly with the information processing unit 4 via the external interface 45.

For example, the in-vivo information acquired by the tag device 1 is received by the RF transceiver 31 and stored in the SRAM 44 in the cell-based IC chip 32 or a dedicated memory provided separately (not shown). The measured data accumulated in memory may be sent later to the information processing unit 4 via the external interface 45. Also, by sending a predetermined request signal later from the information processing unit 4 to the external interface 45 of the relay device 2, it is possible to transmit the measured data accumulated in memory to the main transceiver 3 via the RF transceiver 31. The predetermined request signal may be sent when an explicit instruction is given by the user to the information processing unit 4 or may be sent automatically by the information processing unit 4 on a periodic basis.

Incidentally, the memory for accumulating the in-vivo information may be installed in the tag device 1. In that case, the tag device 1 will transmit to the relay device 2 the in-vivo information accumulated in the memory within itself in response to a request signal received from the information processing unit 4 via the external interface 45 and RF transceiver 31 of the relay device 2. Then, the in-vivo information from the tag device 1 is transferred by the relay device 2 to the information processing unit 4 via the external interface 45.

The power supply 33, which provides operating power to the RF transceiver 31 and cell-based IC chip 32, consists of a cell that can be attached and removed to/from the relay device 2. This cell may be either a primary cell which can no longer be used once all active material has been consumed through chemical reactions or a secondary cell or rechargeable battery which can be reused after recharging. It is also possible to use a battery pack which provides required energy by a combination of two or more rechargeable batteries. The relay device 2, which is attached to the exterior of the body, allows easy battery replacement and charging while causing little strain on the person or animal.

In the configuration of the relay device 2 described above, the RF transceiver 31 and transmit/receive antenna 34 constitute the relay reception means and relay transmission means of the present invention.

Next, description will be given of operation of the in-vivo information extracting system configured as described above. It is assumed that the tag device 1 is left at a desired site within the living body and that the relay device 2 is fastened to the surface of the body near the tag device 1 with adhesive tape or the like.

First, a request signal is transmitted from the main transceiver 3 to the relay device 2 to collect in-vivo information. The request signal may be transmitted either at the request of the information processing unit 4 or by the main transceiver 3 on its own. The request signal received by the RF transceiver 31 is transferred by the relay device 2 from the RF transceiver 31 to the tag device 1. In the tag device 1, the RF transceiver 21 receives the request signal transmitted from the relay device 2 and the power supply 23 generates internal operating power based on an electromagnetic wave in the request signal.

With the operating power supplied from the power supply 23, the tag device 1 sends in-vivo information acquired by the in-vivo information extractor 12 to the relay device 2 via the RF transceiver 21. The relay device 2 receives the in-vivo information via the RF transceiver 31 and transfers it from the RF transceiver 31 to the main transceiver 3. Then, the in-vivo information received by the main transceiver 3 is collected by the information processing unit 4.

This ends one collection operation of in-vivo information. By repeating this operation, it is possible to keep track of changes in in-vivo information with time and utilize the in-vivo information for treatment of the living body, diagnosis, disease control, health care, medical researches, ecological surveys, etc. Incidentally, the in-vivo information received by the main transceiver 3 may be sent to the information processing unit 4 each time it is received or may be accumulated in the main transceiver 3 so that the information processing unit 4 can get it at any desired time.

As described above, according to this embodiment, the operating power needed to drive the tag device 1 is generated internally based on the electromagnetic wave supplied from outside by RFID and the like. Consequently, the tag device 1 does not need to be equipped with a cell or battery and can be downsized accordingly. Specifically, the capsule 5 of the tag device 1 can be downsized to approximately 3 mm in diameter and approximately 10 mm in length, for example.

The tag device 1 can be further downsized if only a small number of sensors are mounted by limiting the type of in-vivo information to be collected. Furthermore, by incorporating sensors into a RFID chip, the tag device 1 can be constituted of only the RFID chip and a transmit/receive antenna. In that case, the tag device 1 can be made sufficiently small because the RFID chip can be configured into a 1-mm square. This makes it possible to reduce inconvenience, discomfort, and pain caused to the person or animal embedded with the tag device 1.

The tag device 1 according to this embodiment is a battery-less type that generates power based on the electromagnetic wave supplied from outside. Thus, once the tag device 1 is embedded in the body, it can be used semi-permanently without replacing it or inserting a lead wire into the body from outside to supply operating power. Here again, it is possible to reduce discomfort and pain caused to the person or animal embedded with the tag device 1.

Since the tag device 1 is very small, it can be swallowed without much discomfort or pain. While the swallowed tag device 1 remains in the stomach or intestine, various in-vivo information can be gathered. Since the tag device 1 is discharged from the body by itself over time, it can be removed from the body without discomfort or pain.

According to this embodiment, signals are exchanged between the tag device 1 and main transceiver 3 via the relay device 2 rather than directly. The tag device 1 generates operating power internally based on the electromagnetic wave supplied from outside. Thus, available power is limited, and so is the range of communication. It is possible to extend the communications range to some extent by increasing the size of the transmit/receive antenna 13, but there is also a limit to this. Besides, the size of the tag device 1 is increased as well.

By equipping the relay device 2 installed near the tag device 1 with the power supply 33, it is possible to increase the communications range between the relay device 2 and main transceiver 3 while enabling short-range communications between the tag device 1 and relay device 2. This makes it possible to deliver high transmit power and thus gain the communications range between the tag device 1 and main transceiver 3 without increasing the size of the tag device 1.

The relay device 2 may also be a battery-less type which generates internal operating power by electromagnetic induction based on the electromagnetic wave sent from the main transceiver 3. In that case, since the relay device 2 installed outside the body is not subject to strict size limitations, the communications range can be extend by using a transmit/receive antenna with high power efficiency. It is preferable to use the power supply 33, which will make it possible to deliver higher power and thus transmit data farther.

If the in-vivo information extracting system described above is used at a hospital, the tag device 1 can be embedded in, or swallowed by, almost any patient without any trouble, whether he/she be slightly ill or gravely ill, and all the conditions of the patient can be controlled centrally by the information processing unit 4 besides treatment of the patients. Also, the tag device 1 can be embedded in a fetus to watch its development or carry out prenatal diagnosis.

Although according to the above embodiment, in-vivo information collection request signals, various commands, etc. are transmitted from the main transceiver 3 to the tag device 1 via the relay device 2, they may be transmitted directly from the main transceiver 3 to the tag device 1. It is when measured data is transmitted from the in-vivo information extractor 12 to the outside that insufficient electromotive force can disable the tag device 1 from conducting long-range communications. However, the main transceiver 3 is capable of transmitting signals even for long distances and the tag device 1 is capable of receiving them. Thus, it is conceivable to transmit in-vivo information collection request signals and the like directly from the main transceiver 3 to the tag device 1 and return acquired in-vivo information from the tag device 1 to the main transceiver 3 via the relay device 2.

Also, although according to the above embodiment, the request signals for collecting in-vivo information, etc. are transmitted from the main transceiver 3 to the tag device 1 via the relay device 2, they may be generated and transmitted to the tag device 1 by the relay device 2 (this corresponds to the second relay transmission means of the present invention). In that case, the relay device 2 can transmit the request signals and the like continuously using a built-in battery.

Also, even if the main transceiver 3 and relay device 2 are not close enough to communicate with each other, it is possible to operate the tag device 1. In that case, however, since the in-vivo information acquired by the in-vivo information extractor 12 of the tag device 1 cannot be transferred from the relay device 2 to the main transceiver 3, measured data should be accumulated in the SRAM 44 in the relay device 2 or a dedicated memory provided separately (not shown).

Then, even if measured data cannot be transferred from the relay device 2 to the main transceiver 3, the measured data accumulated in memory can be sent later to the information processing unit 4 via the external interface 45. Also, by sending a predetermined request signal later from the main transceiver 3 to the relay device 2, it is possible to send the measured data accumulated in memory to the main transceiver 3 from the RF transceiver 31 of the relay device 2.

Incidentally, the memory for accumulating in-vivo information may be installed in the tag device 1 as described above. In that case, the tag device 1 transmits to the relay device 2 the in-vivo information accumulated in the memory within itself to the relay device 2 in response to a request signal supplied directly from the main transceiver 3 or a request signal supplied via the relay device 2. Then, the in-vivo information received from the tag device 1 is transferred by the relay device 2 to the main transceiver 3 via the RF transceiver 31.

In relation to the transmission of an in-vivo information collection request signal and the like from the main transceiver 3, a memory for accumulating in-vivo information may be similarly installed in the tag device 1 or relay device 2. Then, even if a person or animal moves, for example, after transmission of a request signal and the like from the main transceiver 3 to the tag device 1, taking the relay device 2 away from the main transceiver 3 and thus making it impossible to sends in-vivo information in return, the in-vivo information accumulated in the memory can be collectively supplied to the main transceiver 3 and the information processing unit 4, preventing the information processing unit 4 from omitting to collect in-vivo information.

If the memory for accumulating in-vivo information is installed in the relay device 2, when the main transceiver 3 receives the in-vivo information from the relay device 2, an acknowledge signal (Ack signal) will be returned to the relay device 2. If no acknowledge signal is returned within a certain period after the relay device 2 transmits in-vivo information to the main transceiver 3, the in-vivo information accumulated in the memory may be retransmitted.

Similarly, if the memory for accumulating in-vivo information is installed in the tag device 1, when the main transceiver 3 receives the in-vivo information from the tag device 1 via the relay device 2, an acknowledge signal (Ack signal) will be returned to the tag device 1 via the relay device 2. If no acknowledge signal is returned within a certain period after the tag device 1 transmits in-vivo information to the main transceiver 3 via the relay device 2, the in-vivo information accumulated in the memory may be retransmitted.

Then, even if the person or animal moves, making it impossible to transmit in-vivo information from the relay device 2 to the main transceiver 3, attempts to transmit the in-vivo information will be repeated until the in-vivo information is transmitted successfully. This makes it possible to prevent the information processing unit 4 from omitting to collect in-vivo information.

Besides, the embodiments described above are only exemplary of the present invention and are not intended to limit the scope of the present invention. In other words, the present invention can be implemented in various ways without departing from the spirit and major features of the present invention.

As described above, the present invention can downsize tag devices used in the living bodies of people or animals, reducing inconvenience, discomfort, and pain caused to the living bodies. Also, the present invention makes it possible to leave and use tag devices in living bodies for an extended period of time without concern for battery life.

### INDUSTRIAL APPLICABILITY

The present invention is useful in reducing inconvenience, discomfort, and pain caused to the living bodies embedded with tag devices. Also, the present invention is useful in allowing a tag device to be left and used in a living body for an extended period of time without concern for battery life.

1. An in-vivo information extracting system comprising:
a tag device which extracts in-vivo information in a living body; and
a relay device which is installed outside the living body and near the tag device embedded in the living body,
wherein the tag device comprises power generating means for generating internal operating power from an electromagnetic wave fed from outside the tag device, and
the relay device comprises transceiver means for receiving, from the tag device, the in-vivo information extracted by the tag device and transmitting the received in-vivo information to outside the relay device.

2. An in-vivo information extracting system comprising:
a tag device used in a living body, a relay device which is installed outside the living body and near the tag device placed in the living body, and a main transceiver which exchanges signals with the relay device, wherein the tag device comprises:
tag reception means for receiving an electromagnetic wave fed from outside the tag device,
power generating means for generating internal operating power from the electromagnetic wave received by the tag reception means,
in-vivo information extracting means for measuring an environment within the living body and outputting measured data, and
tag transmission means for transmitting the measured data outputted by the in-vivo information extracting means to the relay device; and
wherein the relay device comprises:
relay reception means for receiving the measured data transmitted by the tag device, and
relay transmission means for transmitting the measured data received by the relay reception means to the main transceiver.

3. The in-vivo information extracting system according to claim 2,
the relay device comprises a power supply which is a source of the operating power for the relay reception means and the relay transmission means.

4. The in-vivo information extracting system according to claim 3,
the relay device comprises second relay transmission means for generating and transmitting the electromagnetic wave to the tag device.

5. The in-vivo information extracting system according to claim 2,
the relay device comprises data accumulating means for accumulating the measured data.

6. The in-vivo information extracting system according to claim 5,
the relay transmission means comprises means for transmitting the measured data accumulated in the data accumulating means to outside the relay device in response to a request signal supplied from outside the relay device.

7. The in-vivo information extracting system according to claim 5,
the relay transmission means comprises means for retransmitting the measured data accumulated in the data accumulating means to the main transceiver if no acknowledge signal is returned when the measured data is transmitted to the main transceiver.

8. The in-vivo information extracting system according to claim 2,
the tag device comprises data accumulating means for accumulating the measured data outputted by the in-vivo information extracting means.

9. The in-vivo information extracting system according to claim 8,
the tag transmission means comprises means for transmitting the measured data accumulated in the data accumulating means to the relay device in response to a request signal supplied from outside the tag device.

10. The in-vivo information extracting system according to claim 8,
the tag transmission means comprises means for retransmitting the measured data accumulated in the data accumulating means to the relay device if no acknowledge signal is returned when the measured data is transmitted to the relay device.

11. The in-vivo information extracting system according to claim 2,
the tag reception means and the tag transmission means comprise a low-frequency coil antenna.

12. The in-vivo information extracting system according to claim 2,
the tag reception means and the tag transmission means comprise a radio-frequency planar loop antenna.

13. The in-vivo information extracting system according to claim 2,
the tag reception means and the tag transmission means use a container of the tag device as a radio-frequency antenna.

14. The in-vivo information extracting system according to claim 2,
the relay transmission means transmits control signals to the tag device;
the tag reception means receives the control signals transmitted by the relay transmission means; and
the tag device comprises control means for controlling the in-vivo information extracting means based on the control signals received by the tag reception means.

15. A tag device used for an in-vivo information extracting system which extracts in-vivo information using the tag device placed in a living body and transmits the in-vivo information via a relay device outside the body, the tag device comprises:
tag reception means for receiving an electromagnetic wave fed from outside; and
power generating means for generating internal operating power from the electromagnetic wave received by the tag reception means; and
tag transmission means for obtaining and transmitting measured data about an environment within the living body.

16. The tag device according to claim 15, comprising:
in-vivo information extracting means for measuring the environment within the living body and outputting the measured data,
wherein the tag transmission means transmits the measured data outputted by the in-vivo information extracting means.

17. The tag device according to claim 15, further comprising:
data accumulating means for accumulating the measured data.

18. The tag device according to claim 15,
the tag reception means and the tag transmission means comprise a low-frequency coil antenna.

19. The tag device according to claim 15,
the tag reception means and the tag transmission means comprise a radio-frequency planar loop antenna.

20. The tag device according to claim 15,
the tag reception means and the tag transmission means use a container of the tag device as a radio-frequency antenna.

21. The tag device according to claim 16,
wherein the tag reception means receives control signals transmitted from outside;
and comprising: the tag device comprises control means for controlling the in-vivo information extracting means based on the control signals received by the tag reception means.

22. A relay device used for an in-vivo information extracting system which extracts in-vivo information using a tag device placed in a living body and transmits the in-vivo information via the relay device outside the body, characterized in that the relay device comprises:
relay reception means for receiving measured data about an environment within the living body extracted by the tag device; and
relay transmission means for transmitting the measured data received by the relay reception means.

23. The relay device according to claim 22, further comprising:
a power supply which is a source of operating power for the relay reception means and the relay transmission means.

24. The relay device according to claim 23, further comprising:
second relay transmission means for generating and transmitting an electromagnetic wave in order for the tag device to generate its internal operating power.

25. The relay device according to claim 22, further comprising:
data accumulating means for accumulating the measured data.
